(19) United States
(12) Patent Application Publication
(10) Pub. No.: US 2007/0036851 A1
(43) Pub. Date: Feb. 15, 2007

(54) Acid labile drug compositions

(76) Inventors: Deepak Thassu, West Henrietta, NY (US); Paul Hafey, Victor, NY (US); Leo J. Magee JR., Stafford, NY (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/540,453

(22) PCT Filed: Dec. 9, 2003
(86) PCT No.: PCT/US03/38983

§ 371 Date: Feb. 21, 2006

Related U.S. Application Data
(60) Provisional application No. 60/436,287, filed on Dec. 23, 2002.

(51) Int. Cl. A61K 31/785; A61K 9/20

(52) U.S. Cl. 424/464; 424/78.14

(57) ABSTRACT

A composition and method of making a pharmaceutical dosage form containing an acid labile drug bound to an ion exchange resin is provided. The composition may be coated with a low acid content enteric coating. The binding of the acid labile drug to the exchange resin stabilizes the drug against acid degradation. The low acid content enteric coating controls the permeability of the enteric coating.

### FIELD OF THE INVENTION

This invention relates to acid labile drug compositions.

### BACKGROUND OF THE INVENTION

Proton pump inhibitor drugs are perhaps the best known of the acid labile drug compounds, and include, for example, omeprazole, esomeprazole, lansoprazole, pantoprazole, rabeprazole and leminoprazole.

Acid labile drugs such as the proton pump inhibitors tend to be unstable at acidic pH. For example, even at a relatively mild acidic pH of 6, omeprazole quickly cyclizes and as a result becomes inactive as a drug. This drug degradation can be readily seen in vitro by placing omeprazole in water having a pH of about 6. The water quickly changes color from clear to yellow, then brown, then purple as omeprazole cyclizes and becomes inactive.

As a result of the instability of acid labile drugs, pharmaceutical compounders, have taken steps to protect acid labile drugs from exposure to acid, both during storage and handling, and upon ingestion. Thus, omeprazole drug compositions have been sold in tablet form with an enteric coating. The enteric coating, of course, prevents the tablet from dissolving in the stomach, and thereby prevents the degradation of the omeprazole in the acid environment of the stomach, before it can be absorbed and perform its proton pump inhibitor function.

Unfortunately, omeprazole has proven very sensitive even to the acid content which is typical of enteric coatings. Enteric coatings typically have a potassium hydroxide equivalent acid content of 200-300 mg per gram of coating. In order to protect the omeprazole from the acid contained in the enteric coating, one manufacturer has used a subcoating which separates the omeprazole-containing portion of the tablet from the enteric coating. See U.S. Pat. Nos. 6,207,198 and 6,248,355. Another has additionally used the alkaline salt or an alkaline reacting compound in a core containing the acid labile drug, and coated that with a subcoating. U.S. Pat. Nos. 4,786,505 and 4,853,230.

Of course, the use of a subcoating is a costly process, and makes it difficult to provide omeprazole in stable forms other than tablet form, as for example in liquid formulations.

### SUMMARY OF THE INVENTION

In one aspect of the invention, acid labile drugs are stabilized against acid degradation by conjugating them with ion exchange resins. The resulting stabilization is sufficient to prevent an acid containing enteric coating from reacting with and destabilizing the acid labile drug.

In another aspect of the invention, an enteric coating having a low acid content is used to stabilize an acid labile drug against degradation. A low water permeability polymer may be incorporated into the enteric coating to lower the water permeability of the coating. This provides a time release control capability and also enhances the ability to administer the acid labile drug in suspension form by minimizing permeation of the coating with water used in the suspension.

In yet another aspect of the invention, a powdered drug delivery system is provided which could be used with any active drug capable of conjugating with ion exchange resin particles. The composition is a powder comprising the active drug conjugated to ion exchange resin particles. By adding water or other liquids, one obtains a suspension.

These and other objects, aspects and features of the invention will be more fully understood and appreciated by reference to the written specification.

### DETAILED DESCRIPTION OF PREFERRED EMBODIMENT

In one embodiment of the invention, acid labile drugs are stabilized against acid degradation by conjugating them with ion exchange resin particles. The resulting stabilization is sufficient to prevent an acid containing enteric coating from reacting with and destabilizing the acid labile drug. Conjugation between the acid labile drug and the ion exchange resin particles results from ionic bonds between oppositely charged species because of their mutual electrostatic attraction. Acid labile drugs include drugs that will readily or continually undergo chemical, physical or biological change or breakdown when in an acidic environment. The types of acid labile drugs included in such a preparation include, but are not limited to, omeprazole, esomeprazole, lansoprazole, pantoprazole, rabeprazole and leminoprazole, including complexes of drug bases and salts thereof. These particular drugs are known as proton pump inhibitors, and are used to treat severe erosive esophagitis, gastroesophageal reflux disease (GERD), pathological hypersecretory conditions, peptic ulcer disease and gastric ulcers.

The types of ion exchange resin particles used in the present invention include anionic exchange resin particles. Such anionic exchange resin particles are commercially available as Duolite® resin and resin from Purolite International Limited, both of which are cholestyramnine, a synthetic anionic exchange polymer in which quaternary ammonium groups are attached to a polystyrene-divinylbenzene co-polymer. The resin particles of Duolite® and Purolite are irregularly shaped, i.e., the particles do not conform to geometric shapes such as spherical, elliptical, cylindrical and the like. However, regularly shaped ion exchange resin particles and a combination of irregularly shaped and regularly shaped ion exchange resin particles may be used. Anionic exchange resin particles have cationic moieties that bond with anionic species. Ionic bonding between the anionic exchange resin particles and the acid labile anionic drug results in stabilization of the acid labile drug and prevents an acid containing enteric coating from reacting with and degrading an acid labile drug. The resin may be sized to achieve a desired particle size.

A drug resin conjugate may be prepared by slurrying anionic exchange resin particles in a solution containing an acid labile drug. After the drug complex has formed the complex is washed and dried. The dried resin complex is screened to achieve the desired particle size distribution. A drug loading of 80-95 % is typically obtained with the resin (i.e., 80-95% of the drug is bound to resin), when the drug to resin weight ratio in the slurry is about 1:2. For example, a solution containing 50 mg of omeprazole is slurried with 100 mg of anionic exchange resin particles to form a drug resin complex containing 40-45 mg of omeprazole to 100 mg of anionic exchange resin particles. Of course, the ideal weight ratio of active to resin particles will vary as a function of the charge to weight ratio of the active, and the exchange capacity of the resin particles. To a degree, a greater weight of a higher molecular weight active will bind to a given resin than will a lower molecular weight active, using the same charge per molecule for each active. In the case of omeprazole and this resin, we have found that the maximum amount of omeprazole which can be loaded onto the resin is about one part by weight to two parts by weight resin, or in other words the omeprazole can comprise up to 33% by weight of the final conjugate.

The resin particle size used can vary from about 20 microns to about 125 microns, though within that range preferably varies at least in part as a function of the delivery system to be used. For suspensions, prior artisans have typically used resin particles which as conjugated are 85-110 microns. We have found “as conjugated” particles of 45-80 microns to be preferable for suspensions. For tablets, larger particle sizes can be utilized. However, the particles eventually become so large that they are inefficient, i.e. the weight ratio of active to resin becomes too small. That is because the available bonding charge per unit of weight of resin particle becomes smaller as particles become larger, and more of the charge is buried in the interior of the particle. In other words, the exchange capacity of the resin is diminished.

Preferably, the drug loaded resin particles are coated with an enteric coating having a low acid content. Typical enteric coatings (e.g., copolymers of methacrylic acid and esters of methacrylic acid) have a potassium hydroxide equivalent acid content of 200-300 mg per gram of coating. The low acid content enteric polymers used in the coatings of the preferred embodiment may be a copolymer of phthalic acid such as polyvinyl acetate phthalate or hydroxypropylmethylcellulose phthalate. The low acid content enteric polymers may be used individually or in combination. The low acid content enteric coating ingredient(s) are preferably selected to provide a potassium hydroxide equivalent acid content of under 200 mg per gram of coating, preferably from about 100 mg to about 180 mg per gram of coating. This is accomplished by using a low acid coating enteric polymer having a free acid content of about 1.0% or less. An example of such a low acid enteric coating polymer is a methacrylic copolymer type B having an acid value of about 180 to about 200 mg KOH/gram of dry substance, such as Acrycoat S100. Water permeability of the low acid content enteric coating may be lowered by, for example, blending the low acid content enteric coating with a low permeable polymer such as ethylcellulose. This provides both protection for an acid labile or water sensitive drug, and a time control release capability. It also enhances the ability to administer the acid labile drug in suspension form by minimizing the permeation of the coating with water used in the suspension.

The low acid content enteric coating may also be used to coat a core containing an acid labile drug, wherein the core may comprise drug particles containing an acid labile drug and conventional excipients, such as disintegrants, binders, etc., or to coat drug resin particles comprising an acid labile drug ionically conjugated with an ion exchange resin. The low acid content enteric coating may be coated directly onto the drug particles and/or drug resin complexes without an intervening inert layer, as is conventional. The amount of low acid content enteric coating on the drug resin complex particles depends on several factors, including the particular coating, the desired dosage form, and the desired shelf-life of the dosage form. However, for liquid oral dosage forms comprising low acid content enterically coated drug-resin complex particles, it is desirable to use a thicker coating comprising a blend of a low acid content enteric polymer and a low water permeability polymer, wherein the total weight of the low acid content enteric coating is about 80 to 120% of the weight of the complex. A specific sieve cut of particles to be coated may be used to insure uniform coating of the drug resin complex particles with the low acid content enteric coating.

Using fluid bed technology the drug resin particles may be coated with the low acid content enteric coating. Different amounts of polymer may be used in the low acid content enteric coating and any suitable buffering agent may be added to the dosage form to keep the final pH of the dosage form within a specific range. For example, a specific amount of ethylcellulose may be used in the low acid content enteric coating to control the time release of the drug. For liquid suspensions, a pharmaceutically acceptable buffering agent may be added to maintain about pH 5-6.

The amounts and types of polymers in the low acid content enteric coating and the thickness of the enteric coating applied to the drug resin particles may be selected to control the rate of drug release. The term time controlled release is intended to include sustained release and controlled release drug delivery systems. Sustained release describes the release of a drug substance from a dosage form or delivery system over an extended period of time. Controlled release describes a system in which the rate of drug release is more precisely controlled compared to the sustained release product.

A self-suspending suspension of the present invention may be prepared by slurrying anionic exchange resin particles in a solution containing an acid labile drug to form a drug resin complex. The complex is washed and dried. The dried resin complex is screened to achieve the desired particle size distribution. Surprisingly, a suspending agent is not necessary when drug resin complex particles have a weight average size of about 45-80 μm, even though to form a colloidal suspension, particles have to be 7 microns or less. Prior art suspensions typically use particle sizes of about 85 to about 110 μm. Suspensions using particle sizes this large typically use a suspending agent to keep the particles in a suspended state. In the present invention, a viscosity modifing agent may be used in an amount of approximately 1%. This small amount of a viscosity modifing agent would not be called a suspending agent. Typically, one must use 5-10% of a viscosity modifying agent before it is termed a suspending agent. Alternatively, the screened drug-resin complex particles may be suspended in an oil based suspension to prevent water from getting through the enteric coating of the drug resin particles.

The drug resin complex of the preferred embodiment may be ingested via a variety of pharmaceutical dosage forms. It may be marketed as a suspension. The drug resin complex may be compressed into tablets or caplets. This compressed dosage form may optionally be coated with low acid enteric coating. Such a coating may include a low permeable polymer, i.e., ethylcellulose. The acid labile drug resin complex may also be marketed as a dry powder for oral suspension, i.e., a dry powder to be reconstituted as an oral suspension and dispensed, or reconstituted by the patient and ingested. The dry powder may be reconstituted in a suspension for oral administration to a patient by loosening the powder, such as by lightly tapping a container holding the powder against a hard surface, and then adding an appropriate amount of purified water, or other specified liquid, usually in portions, and shaking the container until all of the dry powder has been suspended. If marketed as a suspension or a reconstitutable powder, it is preferable that the system be buffered to a pH of from about 4 to about 6, using suitable buffering agents. The drug resin complex may be encapsulated in a gelatin capsule, or a capsule of another material, and ingested.

In the foregoing description, it will be readily appreciated by those skilled in the art that modifications may be made to the invention without departing from the concepts disclosed herein. Such modifications are to be considered as included in the following claims, unless these claims by their language expressly state otherwise.

1. An acid labile drug composition comprising:
an acid labile drug conjugated to ion exchange resin particles.

2. The composition of claim 1 wherein the acid labile drug is a proton pump inhibitor.

3. The composition of claim 2 wherein the proton pump inhibitor is omeprazole, esomeprazole, lansoprazole, pantoprazole, rabeprazole or leminoprazole.

4. The composition of claim 1 wherein the ion exchange resin particles are anionic exchange resin particles.

5. The composition of claim 1 wherein the conjugated acid labile drug and resin particles are coated with a low acid content enteric coating.

6. The composition of claim 5 wherein the low acid content enteric coating has a potassium hydroxide equivalent acid content of under 200 mg per gram of coating.

7. The composition of claim 5 wherein the low acid content enteric coating comprises at least one of polyvinyl acetate phthalate, hydroxypropylmethylcellulose acetate phthalate and methacrylic acid copolymer S-100 polymers.

8. The composition of claim 7, wherein the low acid content enteric coating polymer has a free acid content of about 1.0% or less.

9. The composition of claim 8, wherein the low acid content enteric coating further comprises ethylcellulose.

10. A method of making an acid labile drug composition comprising:
slurrying ion exchange resin particles in a solution comprising an acid labile drug and a solvent, thereby creating a drug resin complex; and
washing and drying said drug resin complex.

11. The method of claim 10 wherein the drug complex is sized to achieve a desired particle size.

12. The method of claim 10 wherein the ion exchange resin particles are anionic exchange resin particles.

13. The method of claim 10 wherein the acid labile drug is a proton pump inhibitor.

14. The method of claim 10 wherein the proton pump inhibitor is omeprazole, esomeprazole, lansoprazole, pantoprazole, rabeprazole or leminoprazole.

15. A method of stabilizing an acid labile drug against degradation comprising:
conjugating an acid labile drug with ion exchange resin particles.

16. The method of claim 15 wherein the acid labile drug is a proton pump inhibitor.

17. The method of claim 16 wherein the proton pump inhibitor is omeprazole, esomeprazole, lansoprazole, pantoprazole, rabeprazole or leminoprazole.

18. The method of claim 15 wherein the ion exchange resin particles are anionic exchange resin particles.

19. A composition comprising a core containing an acid labile drug and a low acid content enteric coating disposed directly on the core.

20. The composition of claim 19 wherein the low acid content enteric coating has a potassium hydroxide equivalent acid content less than 200 mg per gram of coating.

21. The composition of claim 19 wherein the acid labile drug is a proton pump inhibitor.

22. The composition of claim 19 wherein the proton pump inhibitor is omeprazole, esomeprazole, lansoprazole, pantoprazole, rabeprazole or leminoprazole.

23. The composition of claim 19 wherein the low acid content enteric coating comprises at least one of polyvinyl acetate phthalate and hydroxypropylmethylcellulose acetate phthalate.

24. The composition of claim 23, wherein the low acid content enteric coating further comprises ethylcellulose.

25. A method of making a self-suspending suspension comprising:
slurrying ion exchange resin particles in a solution comprising an acid labile drug and a solvent, thereby creating a drug complex;
washing and drying said drug complex;
sizing said complex to achieve an average particle size of about 45-80 microns; and
dispersing the particles in a liquid to provide the self-suspending suspension.

26. The method of claim 25 further including a pharmaceutically acceptable buffering agent in an amount effective to maintain a pH of about 5-6.

27. The method of claim 25 further including a small amount of a viscosity modifier.

28. The method of claim 25 wherein the ion exchange resin is an anionic exchange resin.

29. The method of claim 25 wherein the acid labile drug is a proton pump inhibitor.

30. The method of claim 29 wherein the proton pump inhibitor is omeprazole, esomeprazole, lansoprazole, pantoprazole, rabeprazole or leminoprazole.

31. A method of making an acid labile drug suspension comprising:
slurrying ion exchange resin particles in a solution comprising an acid labile drug and a solvent, thereby creating a drug complex;
washing and drying said drug complex; and
suspending said complex in an oil based suspension.

32. The method of claim 31 wherein the drug complex is sized to achieve a desired particle size.

33. The method of claim 31 wherein the resin is sized to achieve a desired particle size.

34. The method of claim 31 wherein the ion exchange resin particles are anionic exchange resin particles.

35. The method of claim 31 wherein the acid labile drug is a proton pump inhibitor.

36. The method of claim 35 wherein the proton pump inhibitor is omeprazole, esomeprazole, lansoprazole, pantoprazole, rabeprazole or leminoprazole.

37. An acid labile drug powder composition for mixing with water or other liquid to form a suspension comprising an acid labile drug conjugated to ion exchange resin particles.

38. The composition of claim 37 wherein the acid labile drug is a proton pump inhibitor.

39. The composition of claim 38 wherein the proton pump inhibitor is omeprazole, esomeprazole, lansoprazole, pantoprazole, rabeprazole or leminoprazole.

40. The composition of claim 37 wherein the ion exchange resin particles are anionic exchange resin particles.

41. A coated acid labile drug composition in which the coating has a low acid content.

42. The composition of claim 41 wherein the coating is an enteric coating.

43. The composition of claim 42 wherein the coating incorporates a low permeability polymer.

44. The composition of claim 41 wherein the coating comprises at least one of polyvinyl acetate phthalate, hydroxypropylmethylcellulose acetate phthalate and methacrylic acid copolymer S-100 polymers.

45. A coated water sensitive drug composition in which the coating is a low acid content enteric coating which also incorporates a low permeability polymer.

46. The composition of claim 45 wherein the coating comprises at least one of polyvinyl acetate phthalate, hydroxypropylmethylcellulose acetate phthalate and methacrylic acid copolymer S-100 polymers.

47. The composition of claim 45 wherein the low permeability polymer is ethylcellulose.

48. A powdered drug composition capable of being reconstituted into a suspension comprising:
an active drug conjugated to ion exchange resin particles.

49. The composition of claim 48 wherein the active drug comprises a proton pump inhibitor.

50. The composition of claim 49 wherein the proton pump inhibitor is omeprazole, esomeprazole, lansoprazole, pantoprazole, rabeprazole or leminoprazole.

51. The composition of claim 48 wherein the ion exchange resin particles are anionic exchange resin particles.

52. A method of treating severe erosive esophagitis, gastroesophageal reflux disease (GERD), pathological hypersecretary conditions, peptic ulcer disease and gastric ulcers comprising administering a drug composition comprising a proton pump inhibitor conjugated to ion exchange resin particles.

53. The method of claim 52 wherein the proton pump inhibitor is omeprazole, esomeprazole, lansoprazole, pantoprazole, rabeprazole or leminoprazole.

54. The method of claim 52 wherein the ion exchange resin particles are anionic exchange resin particles.
